(19) United States
(12) Patent Application Publication
(10) Pub. No.: US 2005/0203543 A1
(43) Pub. Date: Sep. 15, 2005

(54) Surgical guide valve

(76) Inventors: Nabil Hilal, Laguna Niguel, CA (US); Charles C. Hart, Summerville, SC (US)

Correspondence Address: APPLIED MEDICAL RESOUCES CORPORATION, 22872 Avenida Empresa, Rancho Santa Margarita, CA, 92688, US

(21) Appl. No.: 10/802,125

(22) Filed: Mar. 15, 2004

(51) Int. Cl. A61F 11/00

(52) U.S. Cl. 606/108; 604/167.01; 604/167.02; 604/164.02; 604/249; 604/533

(57) ABSTRACT

A surgical access valve is adapted to introduce an instrument into a body cavity or conduit. The valve includes a valve housing and a seal material disposed in the housing. Portions of the seal material define an instrument channel that is normally closed to inhibit fluid communication through the seal material. A dilator is moveable distally to open at least a portion of the instrument channel thereby facilitating passage of the instrument through the seal material, and is also moveable proximally to facilitate formation of a seal around the instrument. The dilator can be biased to a distal position and detented in a proximal position to facilitate one single-handed operation of the valve.

### BACKGROUND OF THE INVENTION

1. Field of the Invention

This invention relates generally to surgical access devices adapted to form a seal in the presence or absence of an instrument extending through the valve.

2. Discussion of the Related Art

Access devices are commonly used with medical catheters to facilitate placement of instruments such as guidewires, laser fibers, fiberoptics, graspers, stent placement devices and the like. These access devices not only facilitate placement of the instruments, but commonly include valves that form seals around the instruments to prevent any retrograde flow of body fluids. In a particularly common use, a valve is placed at the proximal end of an introducer cannula that is inserted into an artery, vein or other body conduit. Various elongate instruments may then be placed, positioned, used or withdrawn through the valve. Many of the instruments are very small in diameter and extremely delicate or flexible. These instruments require that the valve through which they must pass be opened and subsequently closed after the passage of at least the distal, active portion of the instrument.

Touhey-Borst valves have been used for this purpose. These valves comprise two threaded portions that define a cavity adapted to receive an elastomeric material having a central lumen. Unfortunately, this valve requires two-handed operation. As the two threaded portions are relatively twisted in one direction, the elastomeric material is compressed and the lumen is closed. Alternatively, when the threaded portions are relatively twisted in the opposite direction, the material is allowed to relax so that the lumen is open. It will be noted in particular that this elastomeric material is biased to the open position.

The required two-handed operation is particularly cumbersome for most of the procedures that require use of this valve. These procedures often demand that one hand remain on the instrument, leaving only one other hand to operate the valve.

In some procedures, the instrument is highly lubricated or becomes very slippery when wet. Under these conditions, the valve must not only accommodate instrument insertion and sealing, but also provide sufficient traction with the instrument to prevent it from falling out of the access device. With respect to the requirement for traction, the Touhey-Borst valve can be particularly problematical. At a time when the slippery instrument needs to be held, the Touhey-Borst valve requires two-handed operation in order to increase the traction on the instrument.

### SUMMARY OF THE INVENTION

In accordance with the present invention, a surgical access valve is provided with an elastomeric material, such as a gel, having an instrument channel that is normally closed. A dilator is provided with a tubular projection which is adapted to receive the instrument. The dilator is movable from a proximal position to a distal position where the projection extends into the instrument channel of the elastomeric material. This enlarges the channel to receive the instrument. Importantly, movement of the dilator to the distal position can be accomplished using a single hand so that the other hand can be devoted to placement of the instrument.

The dilator may be biased to the proximal position to which it automatically returns upon placement of the instrument. This allows the elastomeric material to move toward its normally closed position, thereby providing a high degree of traction and a seal with the instrument.

In one aspect of the invention, the surgical access valve is adapted to receive an instrument and to form an instrument seal around the instrument. The valve includes a valve housing having an access extending between a proximal end and a distal end, and a seal material disposed in the valve housing. Portions of the seal material define an instrument channel which is normally closed. A dilator is movable distally to open the instrument channel thereby facilitating passage of the instrument through the seal material, and is movable proximally to facilitate formation of the instrument seal around the instrument. The dilator also increases the column strength of a flexible instrument by providing a narrow lumen that inhibits flexing or buckling of the instrument.

In another aspect of the invention, the dilator is movable between a proximal position and a distal position. In the proximal position the dilator is substantially removed from the seal material so that the channel has a first diameter. In the second position, the dilator provides the channel of the seal material with a second diameter greater than the first diameter to facilitate insertion of the instrument. A detent mechanism is disposed between the dilator and the valve housing where it is operable to releasably maintain the dilator in the distal position.

In a further aspect of the invention, finger tabs are provided on the valve housing and the dilator is operable by the thumb of the user to facilitate single-handed operation.

In an associated method of operation, a housing seal is formed between the seal material and the valve housing. The seal material is provided with an instrument channel which is normally closed. A dilator is positioned relative to the seal material and moved at least partially into the instrument channel to facilitate passage of the instrument. This dilator can be removed at least partially from the instrument channel to facilitate formation of an instrument seal between the seal material and the instrument.

These and other features and advantages of the invention will be better understood with a discussion of preferred embodiments of the invention in reference to the associated drawings.

### DESCRIPTION OF THE DRAWINGS

FIG. 1 is a side elevation view of a patient operatively disposed to receive an access device of the present invention;

FIG. 2 is a perspective view of one embodiment of an access device of the present invention in combination with an introducer sleeve or catheter;

FIG. 3 is an enlarged perspective view of the access device illustrated in FIG. 1;

FIG. 4 is a side elevation view of the access device illustrated in FIG. 1;

FIG. 5 is a top plan view taken along lines 4-4 of FIG. 3;

FIG. 6 is a bottom plan view taken along lines 5-5 of FIG. 3;

FIG. 7 is an axial cross-section view taken along lines 6-6 of FIG. 3;

FIG. 8 is an axial cross-section view similar to FIG. 6 and illustrating another embodiment of the present invention;

FIG. 9-11 are axial cross-section views illustrating operation of the embodiment of FIG. 1;

FIG. 9 shows the embodiment in a closed state ready to receive a guidewire;

FIG. 10 shows the embodiment in an open state facilitating insertion of the guidewire; and

FIG. 11 illustrates the embodiment released to its natural state to form a seal and block with the guidewire.

### DESCRIPTION OF PREFERRED EMBODIMENT AND BEST MODE OF THE INVENTION

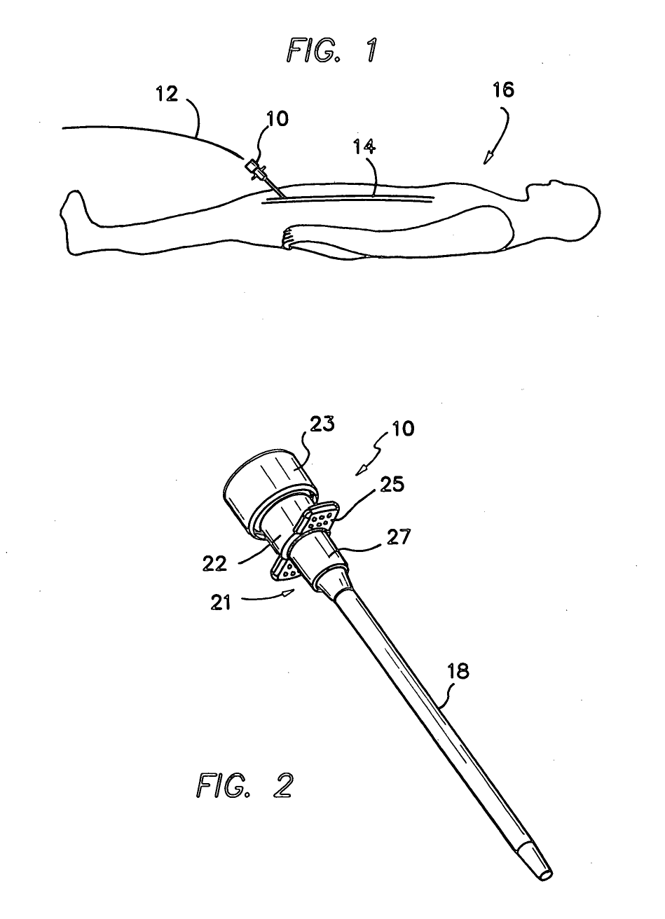

An access device is illustrated in FIG. 1 and designated by the reference numeral 10. This device 10 is intended to facilitate placement of a surgical instrument, access a body wall and into a body conduit or body cavity. Such an instrument might include laser fiber, optical fiber, grasper, stent placement device, or guidewire 12, for example. In use, the access device 10 is inserted into a body cavity or conduit, such as a vein or artery 14, of a patient 16. Once the guidewire 12 has been inserted through the device 10 into the artery 14, it can be advanced to an operative site such as the heart of the patient 16.

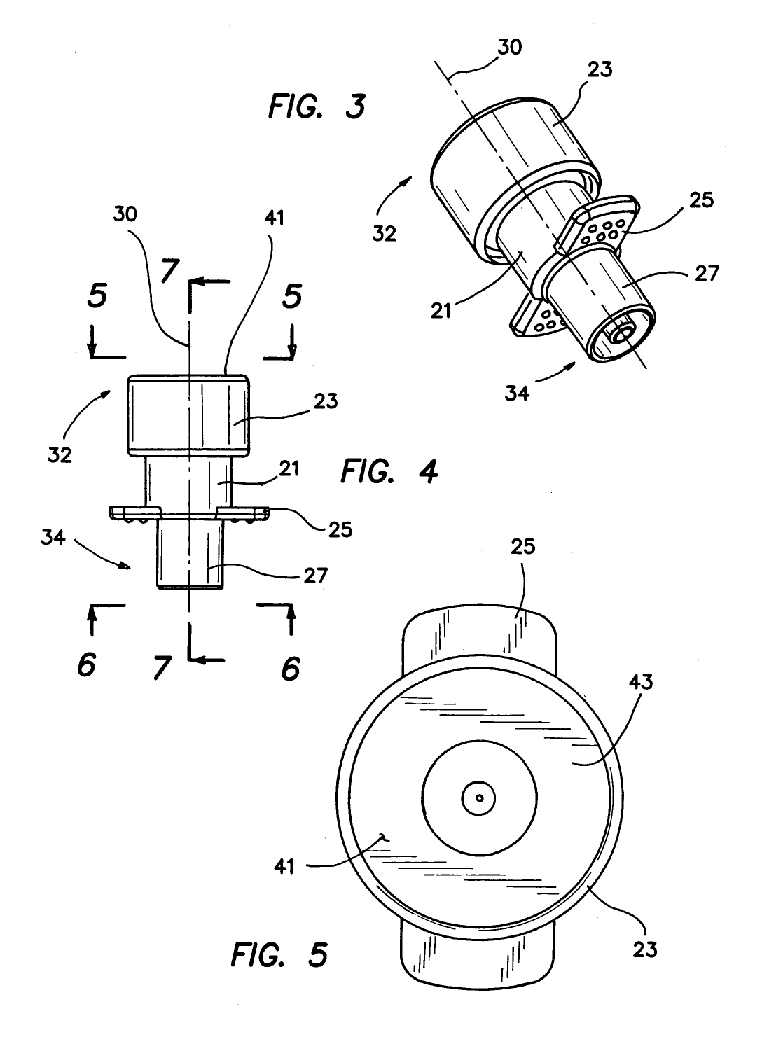

In the enlarged view of FIG. 2, the access device 10 is illustrated in combination with an introducer cannula 18. From this view, and the enlarged views of FIG. 3-7, it can be seen that the access device 10 of this embodiment includes a valve housing 21, a cap or dilator 23, finger tabs 25, and a threaded or fitted connector 27 27 which is best illustrated in FIG. 3. In this view it can be seen that the access device 10 is disposed generally along an axis 30 which extends between a proximal end 32 and a distal end 34.

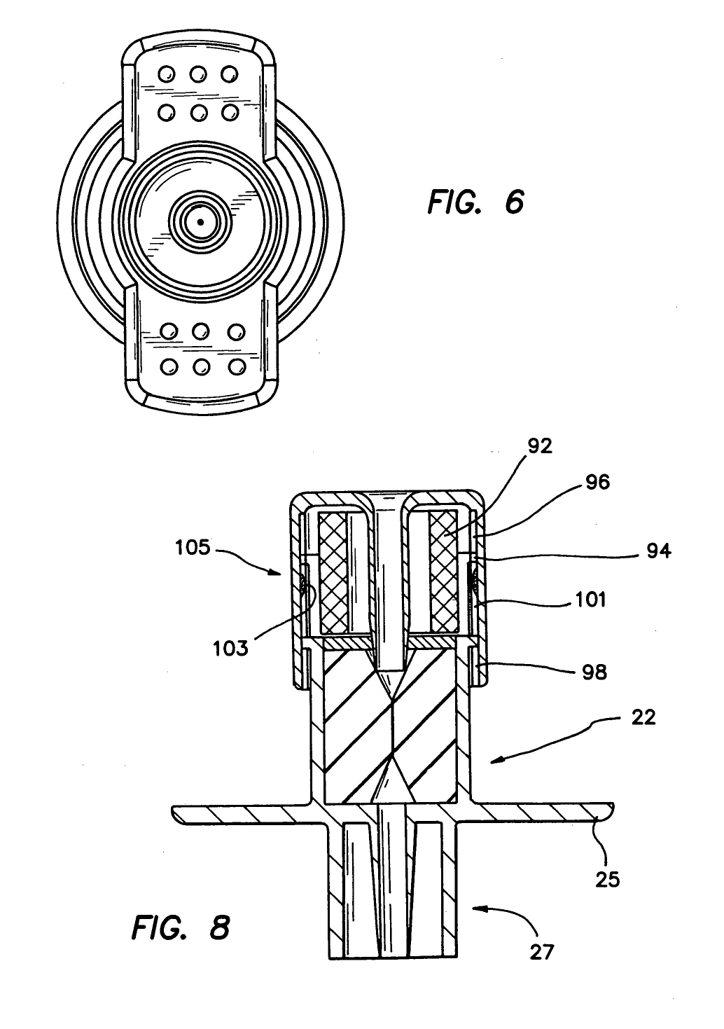

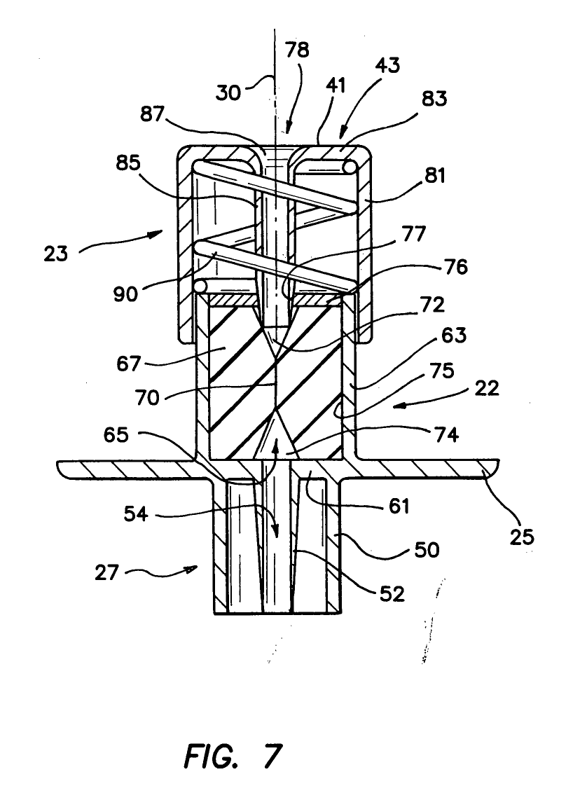

A top surface 41 of the dilator 23 is shown in FIGS. 4 and 5. This surface 41 is generally perpendicular to the axis 30 and functions as a thumb support 43. The connector 27 and finger tabs 25 are best illustrated in the bottom view of FIG. 6. Top surface 41 may incorporate a funnel structure 35, shown in FIG. 7, to facilitate intersertion and centering of the instrument in the lumen of the dilator 23.

The interior regions of the access device 10 are also shown in FIG. 7. In this view it can be seen that the connector 27 includes an outer wall 50, and a central conical projection 52 defining an inner passage 54.

The valve housing 22 includes a bottom wall 61 which in this case is disposed in a plane common with the finger tabs 25. A cylindrical sidewall 63 forms with the bottom wall 61 a valve cavity 65, which is sized and configured to receive a valve 67 of particular interest to the present invention. This valve 67 will typically be formed of a very soft elastomeric material, and configured with a slit 70 disposed along the axis 30. Except for conical shaped voids 70 and 72, this material of the valve 67 fills the valve cavity 65 and forms a housing seal 25 with the housing walls 61 and 63. A retainer 76 having a central hole 77 is disposed to retain the valve 67 in the valve cavity 65.

In a preferred embodiment, the elastomeric material of the valve 67 is a gel material such as that disclosed and claimed by Applicant in U.S. patent application Ser. No. 10/381,220 filed on Mar. 20, 2003 and entitled Surgical Access, which is incorporated herein by reference, in its entirety.

The dilator 23 is positioned generally proximally of the valve housing 22 and in this embodiment includes a cylindrical outer wall 81 and a proximal end wall 83 which functions as the thumb support 43. In this embodiment, a cylindrical projection 85 having a lumen or channel 87 extends axially distally from the end wall 83 concentrically with the outer wall 81. A working channel 78 of the access device 10 extends along the lumen 87 of the dilator 23, the central hole 17 of the retainer 76, the void 72. The slit 70 of the valve 67, the void 74, and the passage 54 of the connector 27 27.

When the access device 10 is assembled, the dilator 23 is positioned proximally of the valve housing 22 with the outer walls 81 of the dilator 23 extending outwardly of the sidewall 63 of the valve housing 22. In this telescoping relationship, the dilator 23 is movable axially, relative to the valve housing 22 between a distal position and a proximal position. In the proximal position illustrated in FIG. 7, the cylindrical projection 85 extends through the hole 77 in the retainer 76 and is positioned generally within the conically shaped void 72.

The dilator 23 is biased to this proximal position by a spring 90 which is supported axially between the cylindrical side wall 63 of the valve housing 22 and the end wall 83 of the dilator 23. This spring 90 functions generally as a means for biasing the dilator 23 to its proximal position.

Structures other than the spring 90 will offer particular advantages in other embodiments where the biasing means may include, for example, an elastomeric material 92, perhaps structured as a foam material and provided in the shape of a cylinder as illustrated in FIG. 8. In this view, the dilator 23 is illustrated in its distal position with the projection 85 extending through the slit 70 of the valve 67. In this distal position, the working channel 8 is defined to a lesser extent by the valve 67. In fact, in the embodiment of FIG. 8, the projection 85 totally opens the slit 70 so that the working channel 78 is defined only by the channel 87 of the projection 85 and the passage 54 of the Connector 27 27, and perhaps a portion of the void 74. With the dilator 23 in this distal position, any instrument inserted into the working channel 78 can avoid major contact with the valve 67. This feature tends to protect the delicate elastomeric material from the instrument and also facilitates axial movement of the instrument without contacting a traction sensitive gel.

In the embodiment of FIG. 8, an annular flange 94, extends outwardly from the proximal end of the valve housing 22. Similar flanges are 96 and 98 extend inwardly from the distal end of the outer wall 81 of the dilator 23. These inwardly extending flanges 96 and 98 define an annular channel 101 within which the flange 94 rides as the dilator 23 is moved between its retracted proximal position and its projected distal position. In this manner, the inwardly extending flanges 96 and 98 associated with the dilator 23 form stops which define the proximal and distal positions of the dilator 23. For example, the flange 96 at the proximal end of the dilator 23 has an interference fit with the annular flange 94 and thereby defines the distal position of the dilator 23 with respect to the valve housing 22. Similarly, the annular flange 98 forms an interference fit with the annular flange 94 when the dilator 23 is in the proximal position.

A projection 103 extending into the channel 101 functions as a detent with the annular flange 94. When the dilator 23 is in its distal position as illustrated in FIG. 8, a retaining element, such as a latch or detent 105, operates to releasably hold the dilator 23 in the distal position. With the biasing means, such as the foam 92 operating to move the dilator 23 to the opposite, proximal position, little force is required to overcome the holding power of the detent 105.

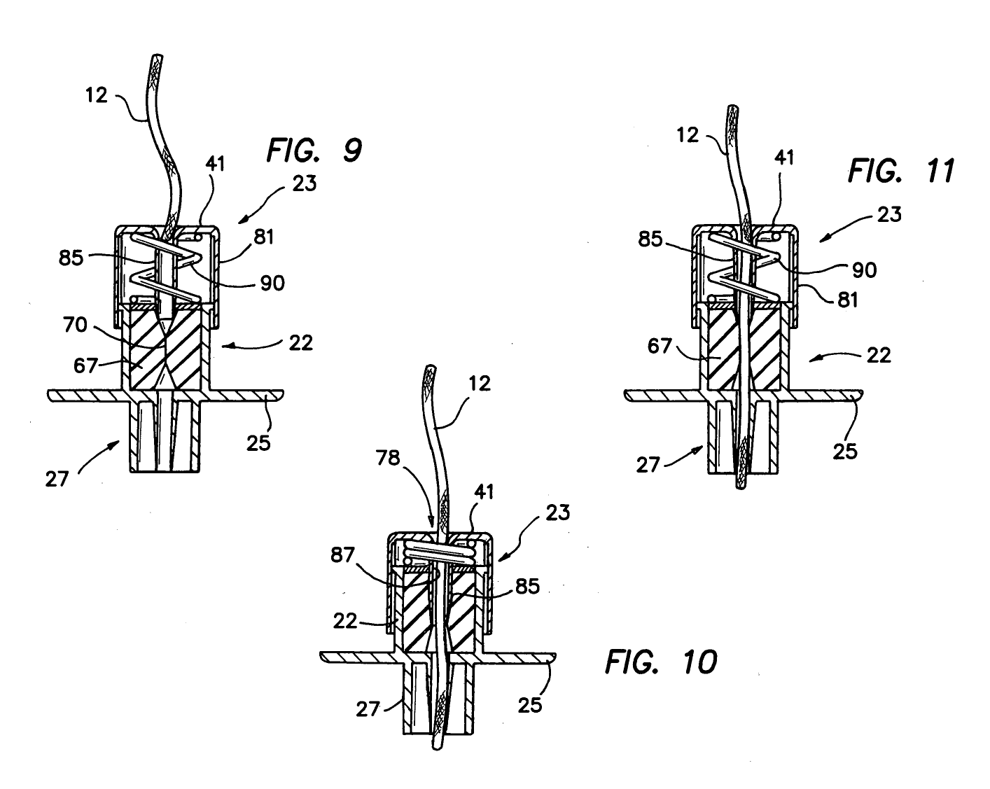

Operation of the access device 10 is best illustrated in the progressive views of FIGS. 9-11. These views show only the access device 10, however, it will be understood that the device 10 is commonly connected to the introducer cannula 18 and inserted into the artery 14 or other body conduit. For example as illustrated in FIG. 1 where the guidewire 12 is intended for insertion into the vessel 14, initially the introducer cannula 18 is inserted into the vessel 14 to provide the desired access.

As illustrated in FIG. 9, the access device 10 is initially disposed with the dilator 23 biased to the proximal position by the spring 90. As previously discussed, in this position the cylindrical projection 85 is retracted from the slit 70 formed in the valve 67. Accordingly, there is no fluid communication through the valve 67. Importantly, with the dilator 23 in its proximal position, the floppy guidewire 12 cannot be inserted through the valve 67 in the access device 10. At this point the user will typically engage the finger tabs 25 with his fingers, and engage the top surface 41 of the dilator 23 with his thumb. Squeezing these elements together, against the bias of the spring 90, will move the dilator 23 to its distal position illustrated in FIG. 10. This movement of course, is accompanied by movement of the cylindrical projection 85 into the slit 70. This tends to dilate or open the valve 67 thereby permitting the floppy guidewire 12 to be passed along the channel 87 and the remainder of the working channel 78.

After the guidewire 12 is inserted, it will typically be of interest to close the valve 67 onto the guidewire 12 in order to hold the guidewire 12 in place. This will also inhibit any retrograde flow of body fluids through the access device 10. As illustrated in FIG. 11, this can be accomplished by merely releasing the access device 10 in which case the spring 90 will automatically force the dilator 23 in the proximal direction. This of course removes the cylindrical projection 85 from the valve 67 allowing the elastomeric material of the valve to automatically expand into contact with the guidewire 12.

Notwithstanding the foregoing distal description, it will be understood that many other modifications can be made to the various disclosed embodiments and method steps, without departing from the spirit and scope of the concept. For example, various sizes of the surgical device are contemplated as well as various types of constructions and materials. It will also be apparent that many modifications can be made to the configuration of parts as well as their interaction. For these reasons, the above description should not be construed as limiting the invention, but should be interpreted as merely exemplary of preferred embodiments. Those skilled in the art will envision other modifications within the scope and spirit of the present invention as defined by the following claims.

1. A surgical access valve adapted to receive an instrument and to form an instrument seal around the instrument, comprising:
a valve housing having an axis extending between a proximal end and a distal end;
a seal material disposed in the valve housing and forming a housing seal with the valve housing;
portions of the seal material defining an instrument channel through the seal material, the instrument channel being normally closed to inhibit fluid communication through the seal material; and
a dilator moveable distally to open at least partially the instrument channel thereby facilitating passage of the instrument through the seal material, and moveable proximally to facilitate formation of the instrument seal around the instrument.

2. The surgical access valve recited in claim 1, wherein the dilator has a channel and is moveable axially between a proximal position and a distal position. The dilator in the distal position, being disposed in the instrument channel of the seal material with the lumen of the dilator extending at least partially through the seal material.

3. The surgical access valve recited in claim 2, further comprising:
means by biasing the dilator to the first position.

4. The surgical access valve recited in claim 3, further comprising:
a retention element disposed between the dilator and the seal housing, the retention element being operable to maintain the dilator in the second position.

5. The surgical access valve recited in claim 1, further comprising:
a thumb support coupled to the dilator; and
at least one finger tab coupled to the seal housing and movable relative to the thumb support to facilitate single-handed movement of the dilator from the proximal position to the distal position.

6. The surgical access valve recited in claim 1, wherein the seal material is a gel.

7. A surgical access valve, adapted to receive an elongate instrument having a column strength, the valve comprising:
a valve housing having an axis extending between a proximal end and a distal end;
a seal material disposed in the valve housing and including portions defining a channel along the axis and through the seal material;
a dilator movable between a proximal position and a distal position;
the dilator in the proximal position being disposed along the axis of the housing;
the dilator in the distal position being disposed distally axially of the proximal position and extending at least partially into the channel; and
the dilator being adapted to receive the elongate instrument and to increase the column strength of the instrument to facilitate passage of the instrument through the seal material.

8. The surgical access valve recited in claim 7, wherein the dilator comprises.
a tube defining a work channel having a width sufficiently narrow to limit lateral binding of the instrument and thereby increases the column strength of the instrument in the working channel.

9. The surgical access valve recited in claim 8, wherein the working channel of the dilator is sized and configured to receive the instrument in the second position of the dilator.

10. The surgical access valve recited in claim 7, wherein the seal material has elastomeric properties.

11. The surgical access valve recited in claim 10, wherein the seal material comprises a gel.

12. The surgical access valve recited in claim 7, further comprising means for biasing the dilator to the proximal position.

13. The surgical access valve recited in claim 12, further comprising means for releasably retaining the dilator in the distal position.

14. The surgical access valve recited in claim 7, further comprising:
a thumb support coupled to the dilator; and
at least one finger tab coupled to the seal housing and movable relative to the thumb support to facilitate single-handed movement of the dilator from the proximal position to the distal position.

15. A surgical access valve, comprising:
a valve housing having an axis extending between a proximal end and a distal end;
a seal material disposed in the valve housing and including portions defining a channel through the seal material;
a dilator movable between a proximal position and a distal position;
the dilator in the proximal position being substantially removed from the seal material so that the channel has a first diameter;
the dilator in the second position providing at least portions of the channel with a second diameter greater than the first diameter to facilitate insertion of the instrument; and
a thumb support coupled to the dilator; and
at least one finger tab coupled to the seal housing and movable relative to the thumb support to facilitate single-handed movement of the dilator from the proximal position to the distal position.

16. The surgical access valve recited in claim 15, wherein the first diameter is substantially zero.

17. The surgical access device recited in claim 15, further comprising:
a retention element disposed between the dilator and the valve housing, the retention element being operable to selectively maintain the dilator in the distal position.

18. The surgical access device recited in claim 15, wherein the dilator has a lumen adapted to receive the instrument in the distal position of the dilator.

19. The surgical access device recited in claim 15, wherein the seal material has elastomeric properties.

20. The surgical access device recited in claim 19, wherein the seal material comprises a gel.

21. The surgical access device recited in claim 15, wherein the dilator is biased to the proximal position.

22. The surgical access device recited in claim 21, wherein the seal material is biased to the first diameter.

23. A surgical access valve adapted to receive an instrument and to form an instrument seal around the instrument; comprising:
a valve housing having an axis extending between a proximal end and a distal end;
a seal material forming a single valve in the valve housing;
portions of the seal material defining an instrument channel, the portions forming a zero seal in the absence of the instrument and the instrument seal in the presence of the instrument;
a dilator movable from a proximal position to a distal position thereby enlarging at least a portion of the instrument channel to facilitate insertion of the instrument; and
the dilator being movable from the distal position to the proximal position to facilitate formation of the instrument seal between the seal material and the instrument.

24. The surgical access valve recited in claim 23, wherein the seal material has elastomeric properties.

25. The surgical access valve recited in claim 24, wherein the seal material is a gel.

26. The surgical access valve recited in claim 23, further comprising:
means for biasing the dilator to the proximal position.

27. The surgical access valve recited in claim 23, wherein the seal material is biased toward formation of the zero seal.

28. The surgical access valve recited in claim 23, further comprising:
a retention mechanism disposed between the dilator and the valve housing.

29. The surgical access valve recited in claim 23, further comprising:
a thumb support coupled to the dilator; and
at least one finger tab coupled to the seal housing and movable relative to the thumb support to facilitate single-handed movement of the dilator from the proximal position to the distal position.

30. A method for operating a surgical access valve having a valve housing with an access extending between a proximal end and a distal end, comprising the steps of:
placing a seal material within the valve housing;
forming a housing seal between the valve housing and the seal material;
forming an instrument channel through the seal material, the instrument channel being normally closed to inhibit fluid communication through the instrument channel;
positioning a dilator relative to the seal material and the instrument channel;
moving the dilator at least partially into the instrument channel to facilitate placement of an instrument in the instrument channel; and
removing the dilator at least partially from the instrument channel to facilitate formation of an instrument seal between the seal material and the instrument.

31. The method recited in claim 30, further comprising the step of:
providing the seal material with properties for biasing the instrument channel to a normally closed state.

32. The method recited in claim 31, further comprising the step of:
biasing the dilator to facilitate the removing step.

33. The method recited in claim 32, further comprising the steps of:
during the moving step, moving the dilator distally to an instrument insertion position; and
releasably holding the dilator at the instrument insertion position.

34. The method recited in claim 33, wherein the moving step comprises the step of:
using only a single hand to move the dilator to the instrument insertion position.
